(19) United States
(12) Patent Application Publication
(10) Pub. No.: US 2006/0184091 A1
(43) Pub. Date: Aug. 17, 2006

(54) Liquefaction handpiece

(75) Inventors: Ramon C. Dimalanta, Rancho Santa Margarita, CA (US); Glenn Sussman, Laguna Nigel, CA (US)

Correspondence Address: ALCON, IP LEGAL, TB4-8, FORT WORTH, TX, 76134-2099, US

(73) Assignee: Alcon, Inc.

(21) Appl. No.: 11/057,515

(22) Filed: Feb. 14, 2005

(51) Int. Cl. A61N 1/30

(52) U.S. Cl. 604/19

(57) ABSTRACT

A liquefaction handpiece having an internal or integral reservoir for the working fluid. The reservoir may either feed the heater/pump by gravity or may use capillary or wicking action to feed the heater pump.

### BACKGROUND OF THE INVENTION

This invention relates generally to the field of cataract surgery and more particularly to a handpiece for practicing the liquefaction technique of cataract removal.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted with the injection of the heated solution and the injection of a relatively cool solution, thereby quickly cooling and removing the heated solution. This technique is more fully described in U.S. Pat. No. 5,616,120 (Andrew, et al.), the entire contents of which is incorporated herein by reference. The apparatus disclosed in the publication, however, heats the solution separately from the surgical handpiece. Temperature control of the heated solution can be difficult because the fluid tubings feeding the handpiece typically are up to two meters long, and the heated solution can cool considerably as it travels down the length of the tubing.

One handpiece that heats the working fluid internally is described in U.S. Pat. No. 6,398,759 B1 (Sussman, et al.) and is commercially available from Alcon Laboratories, Inc., Fort Worth, Tex. Other handpieces are described in U.S. Pat. Nos. 5,865,790 and 6,527,766 (both to Bair) and U.S. Pat. No. 6,440,103 (Hood, et al.). These handpieces all require an external source of the working fluid.

Therefore, a need continues to exist for a surgical handpiece that has an integral source for the fluid solution used to perform the liquefaction technique.

### BRIEF SUMMARY OF THE INVENTION

The present invention improves upon the prior art by providing a liquefaction handpiece having an internal or integral reservoir for the working fluid. The reservoir may either feed the heater/pump by gravity or may use capillary or wicking action to feed the heater pump.

Accordingly, one objective of the present invention is to provide a handpiece suitable for practicing the liquefaction technique of lens removal.

Another objective of the present invention is to provide a handpiece suitable for practicing the liquefaction technique of lens removal and having an integral source for the working fluid.

These and other advantages and objectives of the present invention will become apparent from the detailed description and claims that follow.

### BRIEF DESCRIPTION OF THE DRAWINGS

FIG. 1 is an enlarged cross-sectional view of a first embodiment of the handpiece of the present invention.

FIGS. 2A-2B are enlarged cross-sectional views of a first embodiment of the handpiece of the present invention.

FIGS. 3A-3B are enlarged cross-sectional views of a second embodiment of the handpiece of the present invention.

### DETAILED DESCRIPTION OF THE INVENTION

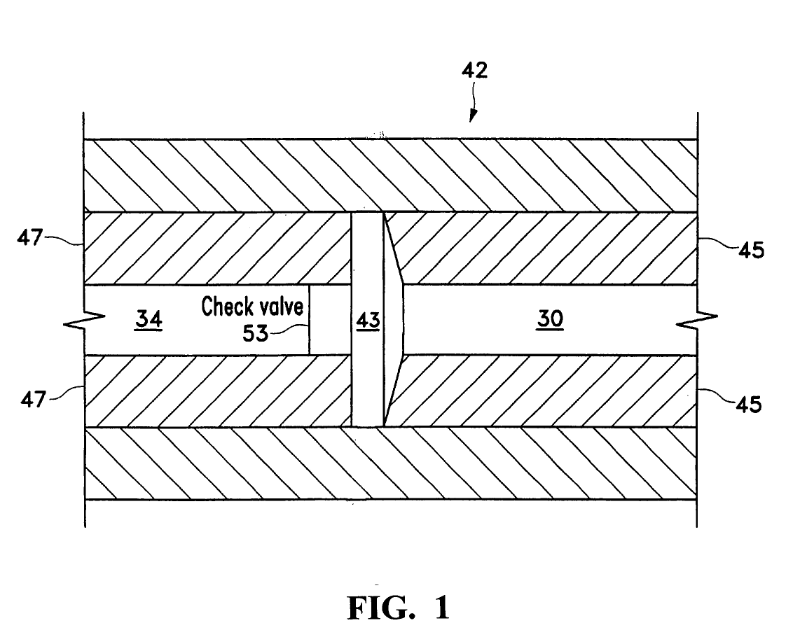

As best seen in FIG. 1, in general, pumping chamber 42 contains a relatively large pumping reservoir 43 that is sealed on both ends by electrodes 45 and 47. Electrical power is supplied to electrodes 45 and 47 by insulated wires, not shown. In use, surgical fluid (e.g. saline irrigating solution) enters reservoir 43 through tube 34 and check valve 53, check valves 53 being well-known in the art. Electrical current (preferably Radio Frequency Alternating Current or RFAC) is delivered to and across electrodes 45 and 47 because of the conductive nature of the surgical fluid. As the current flows through the surgical fluid, the surgical fluid boils. As the surgical fluid boils, it expands rapidly out of pumping chamber 42 through tube 30 (check valve 53 prevents the expanding fluid from entering tube 34). The expanding gas bubble pushes the surgical fluid in tube 30 downstream of pumping chamber 42 forward. Subsequent pulses of electrical current form sequential gas bubbles that move surgical fluid down tube 30. The size and pressure of the fluid pulse obtained by pumping chamber 42 can be varied by varying the length, timing and/or power of the electrical pulse sent to electrodes 45 and 47 and by varying the dimensions of reservoir 43. In addition, the surgical fluid may be preheated prior to entering pumping chamber 42. Preheating the surgical fluid will decrease the power required by pumping chamber 42 and/or increase the speed at which pressure pulses can be generated.

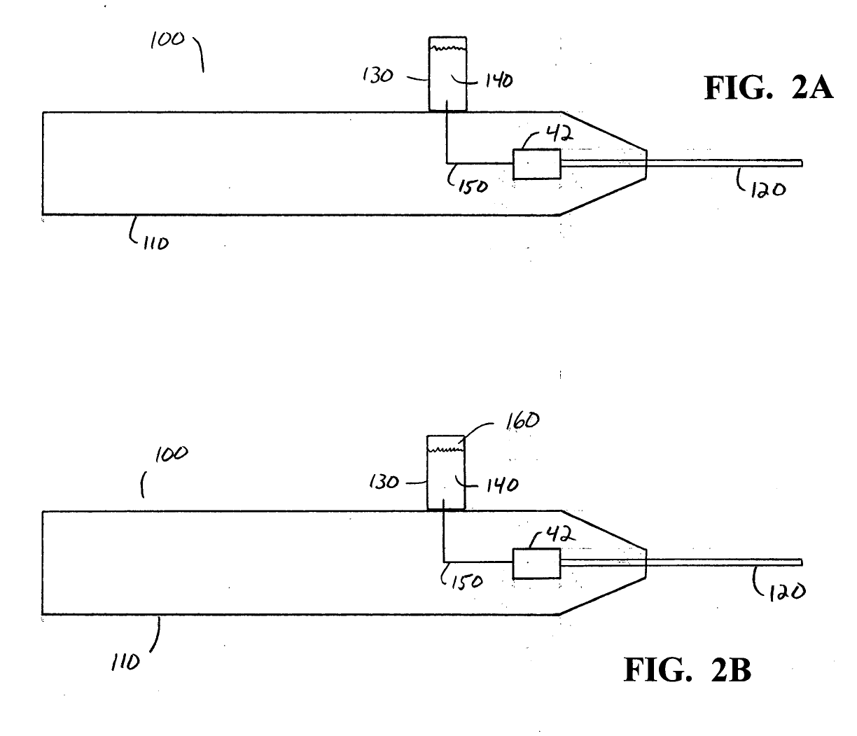

In a first embodiment of the handpiece of the present invention, seen in FIGS. 2A-2B, handpiece 100 has body 110 containing pumping chamber 42 connected to tip 120 in the manner generally described in U.S. Pat. No. 6,579,270 B2 (Sussman, et al.). Tip 120 may have a construction similar to the tips described in this reference, FIGS., 23 and 24 and the discussion at column 7, lines 31-45 of U.S. Pat. No. 6,579,270 B2 specifically being included by reference. Attached to or integrally formed within body 110 is fluid reservoir 130, containing a surgical irrigating solution, or liquefaction working fluid 140. Fluid 140 is supplied to pumping chamber 42 by fluid line 150. Handpiece 100 in FIG. 2A relies on gravity pressure to feed fluid 140 to pumping chamber 42 while handpiece 100 in FIG. 2B relies on pressurized gas 160, such as CO₂, to force fluid 140 through line 150.

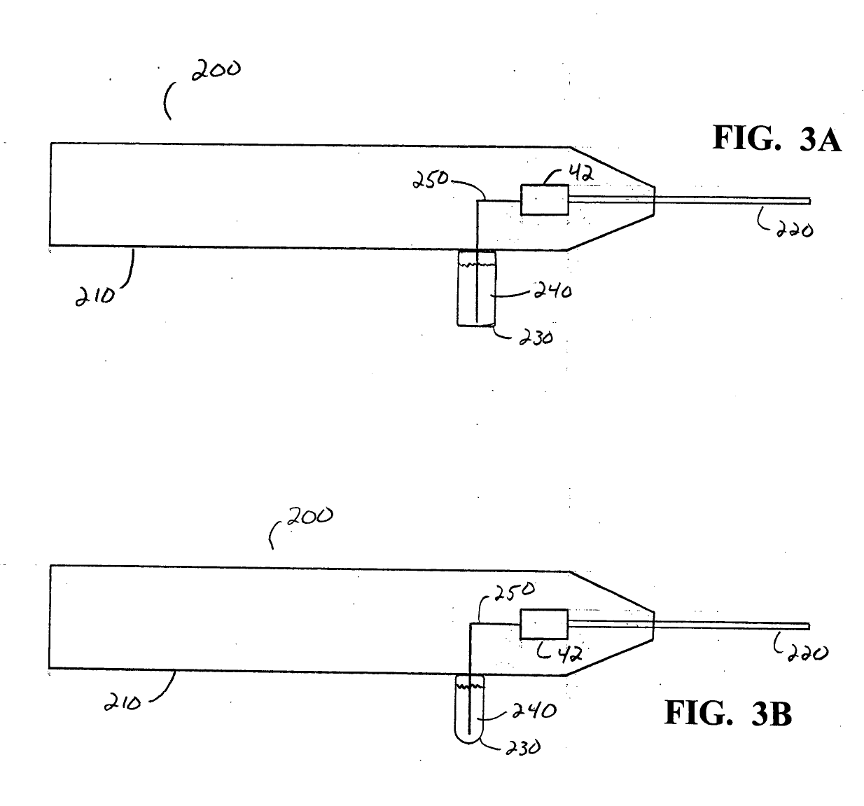

In a second embodiment of the handpiece of the present invention, seen in FIGS. 3A-3B, handpiece 200 has body 210 containing pumping chamber 42 connected to tip 220 in the manner generally described in U.S. Pat. No. 6,579,270 B2 (Sussman, et al.). Tip 220 may have a construction similar to the tips described in this reference, FIGS., 23 and 24 and the discussion at column 7, lines 31-45 of U.S. Pat. No. 6,579,270 B2 specifically being included by reference. Attached to or integrally formed within body 210 is fluid reservoir 230, containing a surgical irrigating solution, or liquefaction working fluid 240. Fluid 240 is supplied to pumping chamber 42 by fluid line 250. Fluid reservoir 230 may be a rigid container, as shown in FIG. 3A, or a collapsible bag, as shown in FIG. 3B. Fluid 240 flows to pumping chamber 42 by capillary or wicking action through line 250.

This description is given for purposes of illustration and explanation. It will be apparent to those skilled in the relevant art that changes and modifications may be made to the invention described above without departing from its scope or spirit.

We claim:

1. A liquefaction handpiece, comprising:
a) a body;
b) a pumping chamber in the body and fluidly connected to a tip; and
c) a fluid contained formed in or on the body and fluidly connected to the pumping chamber.

2. The handpiece of claim 1 wherein fluid flows from the container to the pumping chamber by gravity pressure.

3. The handpiece of claim 1 wherein fluid flows from the container to the pumping chamber by gas pressurization.

4. The handpiece of claim 1 wherein fluid flows from the container to the pumping chamber by capillary action.

5. A handpiece of claim 1 wherein fluid flows from the container to the pumping chamber by wicking action.
